(19) United States
(12) Patent Application Publication
(10) Pub. No.: US 2015/0305631 A1
(43) Pub. Date: Oct. 29, 2015

(54) Real-Time Relationship Between Geometries of an Instrument and a Structure

(71) Applicant: Medtronic, Inc., Minneapolis, MN (US)

(72) Inventors: Ryan P. Lahm, Lino Lakes, MN (US); Melissa Gene Tanner Christie, Andover, MN (US); Eric A. Schilling, Ham Lake, MN (US)

(73) Assignee: Medtronic, Inc., Minneapolis, MN (US)

(21) Appl. No.: 14/262,027

(22) Filed: Apr. 25, 2014

(51) Int. Cl. A61B 5/02; A61B 5/00

(52) U.S. Cl. CPC A61B 5/02007; A61B 5/7275; A61B 2560/0487

(57) ABSTRACT

A system is disclosed for illustrating a geometry of a structure and a geometry of an instrument. The system can implement a set of instructions to assist in determining an appropriateness of positioning a selected instrument at a location. The system may display the geometry of the structure and/or the geometry of the instrument.

### FIELD

The subject disclosure relates to a system for illustrating a device, and particularly for illustrating a device relative to a diameter of a vessel.

### BACKGROUND

This section provides background information related to the present disclosure which is not necessarily prior art.

During various procedures, an instrument can be positioned within a volume of a vessel. The vessel can include a vasculature, such as a vein, of a patient. The vessel of the patient may include a diameter along its length. The instrument positioned within the vessel may include a lead, such as a stimulation or cardiac rhythm lead. The lead may generally be held within the vessel based upon an interference fit of the lead within the vessel.

### SUMMARY

Further areas of applicability will become apparent from the description provided herein. The description and specific examples in this summary are intended for purposes of illustration only and are not intended to limit the scope of the present disclosure.

In a selected procedure, an instrument can be positioned within a structure. The structure may be any appropriate structure into which an instrument may be placed. For example, the structure may include a vasculature of a patient, tubing of a system, or pipes of a sewer system. The instrument may be any appropriate instrument as well. For example, the instrument may include an optic camera, such as one to view an interior of a water passage system, oil passage system, or other exemplary system. For example, a user may select to investigate an integrity of a pipe system that may be included in an architectural structure, a cooling system (e.g. heat exchanger) for a vehicle or engine, or an oil transport line. The investigative device can include a geometry, such as a diameter. The geometry of the instrument may be determined to fit within interior wall surfaces of the pipe or passage system. Accordingly, a selection system and/or method can be used to ensure or select an appropriate size of the investigative instrument.

According to various embodiments, the instrument may include a lead. The lead can include a lead for a cardiac resynchronization system. The lead may also include a lead for a stimulation system, such as a nervous stimulation system. In addition, the instrument can include a pulmonary instrument that may include sensors such as pressure sensors and/or position sensors.

A selection or suggestion system can include information that is analyzed to determine a geometry of a structure, such as a tubing or pipe system, although the structure may include a vasculature of a subject. The analysis can be performed on a model that is generated or based upon image data acquired of the subject. The model can be a selected model, such as a two-dimensional (2D) model and/or a three-dimensional (3D) model. The analysis of the geometry of the passage system, such as a vasculature, can be used to identify a diameter at various arc-like segments over a selected length of the vasculature.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a detail view of a structure system;

FIG. 2 is a schematic illustration of a plot of a diameter of a structure over a length of the structure;

FIG. 3 is an illustration of a screenshot of a plot of a diameter of a structure over a plurality of arclengths relative to a plot of instrument diameters, according to various embodiments;

FIG. 4 is an illustration of a screenshot of a plot of a diameter of a structure over a plurality of arclengths relative to a plot of instrument diameters, according to various embodiments;

FIG. 5 is an illustration of a screenshot of a plot of an envelope diameter of a structure over a plurality of arclengths relative to a plot of instrument diameters, according to various embodiments;

FIG. 6 is an illustration of a system configured to operate with the disclosed system, according to various embodiments; and

FIG. 7 is a flowchart of a method for illustrating a selected instrument geometry relative to a structure geometry.

Corresponding reference numerals indicate corresponding parts throughout the several views of the drawings.

### DETAILED DESCRIPTION

Example embodiments will now be described more fully with reference to the accompanying drawings.

In a physical structure, a passage may be bounded by a surface formed by a wall. The surface formed by a wall can include a pipe structure, such as in tubes of a heat exchanger, pipe passages in a building structure, or a vasculature of a patient. In a selected physical structure it may be selected to position an instrument for various procedures. The procedure may include moving a viewing instrument, such as a fiber optic camera, to inspect a pipeline in a building structure or heat exchanger for issues, such as leaks or cracks. Also, a procedure may include positioning of a lead for implantation within a subject. The lead can include a cardiac rhythm lead, pressure sensing lead, or other implantable instrument. Nevertheless, according to various embodiments, including those discussed herein, it may be selected to identify the geometry, including at least an internal diameter, of a passage for determining an appropriate size, path, and other features of positioning an instrument. It may be selected, for example, to obtain analysis such as image analysis of a structure to determine the geometry of the passages.

It is understood that the following disclosure may relate to positioning an instrument within a subject, such as a human subject, however, an instrument can be positioned within any appropriate system, such as tubes in a heat exchanger, water pipes in a structure, or other appropriate system. Further, although the following example may relate to positioning a cardiac lead within the vasculature of a patient, such as a cardiac resynchronization lead, other appropriate instruments may be used. For example, a pressure sensor, an ablation instrument, or the like may be incorporated into an instrument. It is understood that the system discussed herein may relate to such instruments.

In one example, a lead can include any appropriate lead that can be delivered to a patient. For example, the cardiac resynchronization therapy (CRT) leads can include the Attain Ability® leads and the Attain STARFIX® leads that can be interconnected with a selected resynchronization implant (generally referred to as an implanted medical device (IMD)). IMDs can include the VIVA XT® IMD or the Protecta® XT CRT-D IMD sold by Medtronic, Inc. The Attain® leads can be a part of the Attain® CRT implant system sold by Medtronic, Inc., having a place of business in Minnesota, U.S.A. Further, additional portions could be used to assist in positioning the leads such as an Attain Command® catheter or catheters and one or more Attain Select® II sub-selection catheters. All of these systems can be passed through vasculature of the patient prior to implanting the lead within the patient. Further, the lead may be held within the patient at a selected location that is based upon an interaction of the lead with the anatomy of the patient, such as a wall of the vasculature.

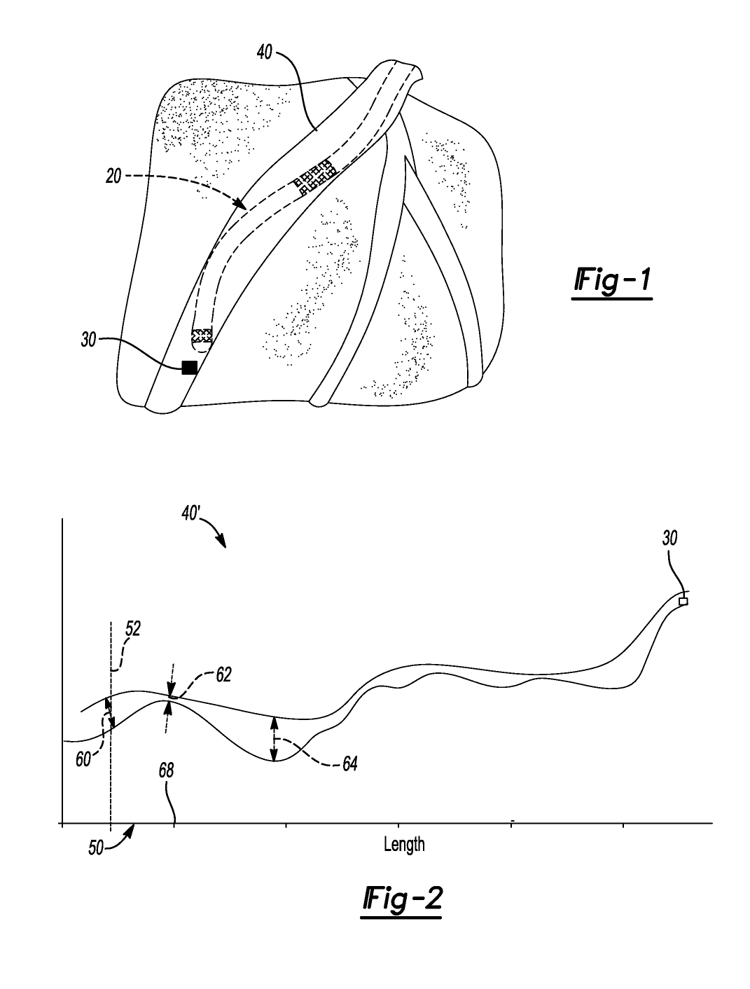

For example, as illustrated in FIG. 1, a lead 20 may be selectively positioned relative to a selected target location 30 within a selected or target structure, such as a vessel 40 of the subject. In an exemplary system, the vessel 40 may be a left ventricular vessel, such as a branch from a coronary sinus. Positioning of the lead 20 within the coronary sinus or a branch thereof is generally understood by one skilled in the art, and the procedural details will not be described in detail here. Nevertheless, the presently disclosed system can be used to assist in suggesting and/or ranking possible leads to be positioned near or at the target location 30 within the vessel 40.

Various systems can be used to determine or analyze the anatomy or geometry of a vessel or passage system. For example, the CardioGuide® System sold by Medtronic, Inc. can obtain image data or analyze image data of a patient. The image data can be used to generate a 2D or 3D model of the geometry of a vessel over a length can be analyzed and/or determined. In addition thereto, or alternatively thereto, other systems can be used to analyze the geometry of vessels of a subject or other appropriate system. For example, magnetic resonance image data (MRI data) could be used to determine the geometry of a portion of a subject. Further, any appropriate venogram image data system can be used to generate image data that can be analyzed in an appropriate manner. Further, certain systems, such as those disclosed in U.S. patent application Ser. No. ______ (Attorney Docket No. 5074D-000086-US) entitled, “A METHOD AND SYSTEM FOR RANKING INSTRUMENTS” describes a system that can be used to analyze image data, incorporated herein by reference.

With reference to FIG. 2, a schematic illustration of the vessel 40 is illustrated. The vessel 40 is schematically and exemplarily illustrated on a graph or relative to a line 50 representing length or a distance from an insertion or initial point 52 to the target 30. Along the length line 50, the vessel 40 may have varying diameters, such as a first diameter 60 and a second diameter 62, and a third diameter 64. It is understood that the diameter of the vessel 40 may be determined at any appropriate segmented length along the total length 50 of the vessel 40. Each of the individual lengths may be referred to as “arc lengths” as the vessel 40 may not be elongated along only a substantially straight line. Accordingly, each length segment along the length 50 of the vessel 40 may be referred to as an arc length. It is understood, however, that the structure being analyzed may not be a circle or perfectly circular under all circumstances. Thus, the geometry analyzed and determined may be a different regular or irregular geometric shape. Herein, a diameter may refer to a cross-sectional dimension, including a greatest cross-section dimension of the analyzed structure.

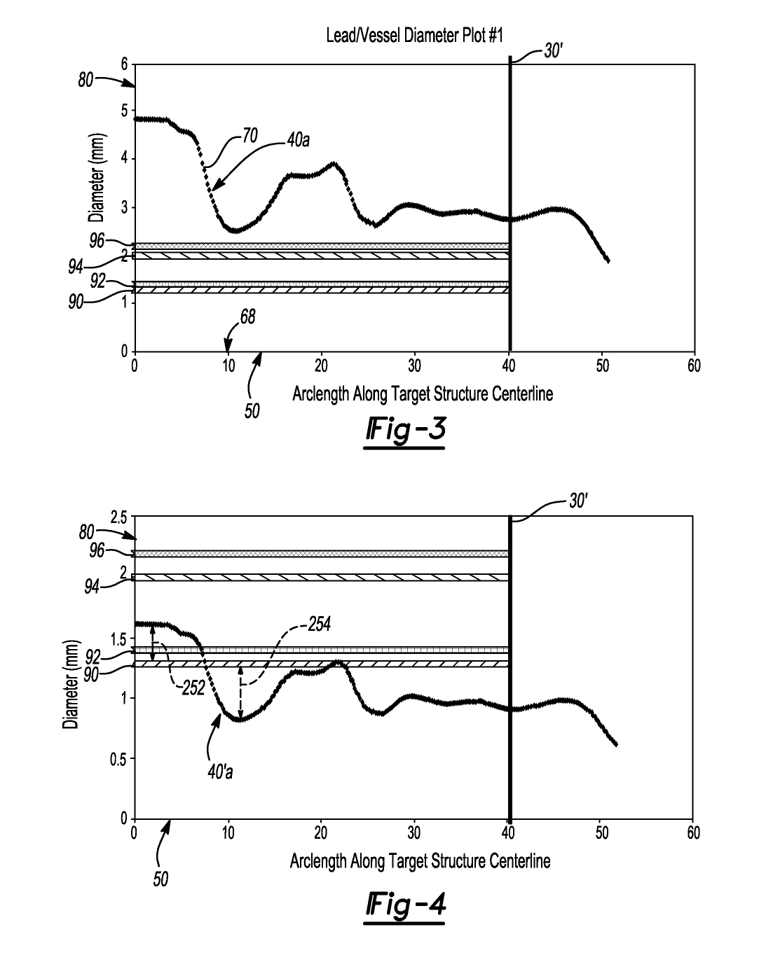

As exemplarily illustrated in FIG. 2, a plot of the vessel may illustrate each arc length between hash marks 68 illustrated along the length line 50. Each arc length can be analyzed to determine a diameter of the vessel 40, as discussed further herein. The diameter along the length of the vessel 40 may be summed over a total length and illustrated at each arc length, as exemplarily illustrated in FIG. 3.

As illustrated in FIG. 3, the length line 50 includes hash marks 68 that show the cumulative arc lengths along the length line 50, including 10 mm, 20 mm, etc. The summation of the arc lengths can be along a center line of the vessel. As is also illustrated in the chart in FIG. 3, a diameter at each arc length segment can be illustrated by a selected symbol, such as a diamond 70. A vertical axis 80 can illustrate or represent the diameter such that the symbols 70 can be plotted on the horizontal axis length line 50 and the vertical axis diameter line 80 to illustrate the diameter of the vessel 40 along its cumulative arc lengths.

As illustrated in FIG. 3, the vessel 40 is illustrated as a plurality of symbols to identify the diameter of a specific arc length portion of the vessel 40 over its total cumulative length illustrated along the axis 50. The graph including the vessel line 40a illustrates the diameter of the vessel 40 along its length and how the diameter varies along the length. Plotted on the same chart relative to the horizontal axis 50 and the vertical axis 80 can be one or more lead lines 90, 92, 94, and 96. The lead lines 90-96 can illustrate different leads that may possibly be positioned within the vessel for a selected procedure. For example, as illustrated in FIG. 3, the lead lines 90-96 relate to respective diameters of different leads. For example, lead lines 90 and 92 are between about 1 mm and 1.5 mm in diameter. Lead lines 94 and 96 represent leads that are about 2 mm in diameter. Accordingly, the diameter of the various leads can be illustrated relative to or scaled to the diameter of the vessel 40 along its length as illustrated by the vessel chart plot 40a.

With reference to FIG. 4, a vessel 40′ may be illustrated as a vessel plot 40′a on the graph including the two axes 50, 80; similar to the chart illustrated in FIG. 3. The vessel 40′ illustrated in the chart in FIG. 4, however, can include a different diameter along its various arc lengths as illustrated in FIG. 4. Again, the lead lines 90-96 can be illustrated on the same chart to illustrate their respective diameters relative to the diameter of the vessel plot 40′a.

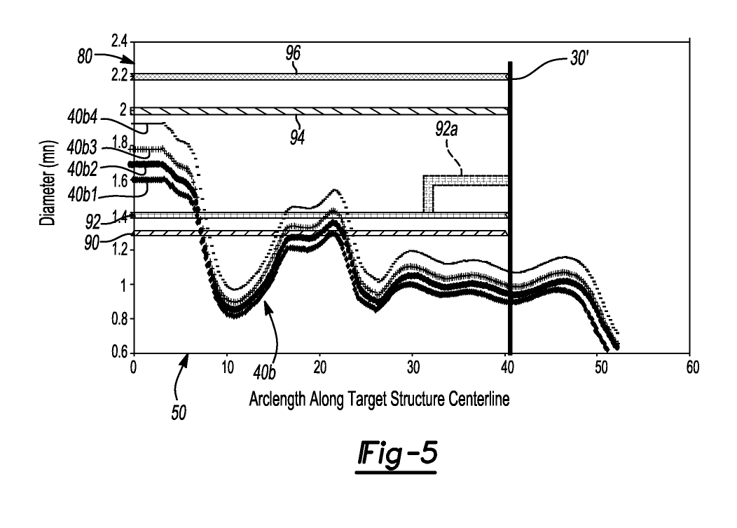

With reference to FIG. 5, a plot of a vessel, such as the vessel 40, can be illustrated as vessel plot 40b. The plot 40b can include several plots such as 40b1 that illustrates a nominal or measured (e.g. determined by image or model analysis) diameter of a vessel plotted on the axes 50 and 80. The graph can further include various stretch factors, such as a 5% stretch plot 40b2, a 10% stretch factor plot 40b3 and a 20% stretch factor plot 40b4. Each of the stretch factors can represent a diameter based upon a stretch of the vessel of 5%, 10% or 20% relative to the determined or measured nominal diameter illustrated by the plot 40b1. According to various embodiments, therefore, as illustrated in FIG. 5, a determination of a stretch factor or a stretch diameter can also be determined and plotted relative to the axes 50 and 80. Again, the illustration of the lead lines 90-96 can also be plotted on the same axes relative to the stretch factor plots 40b1-40b4 to illustrate the diameter of the lead scaled relative to the stretch diameter of the vessel.

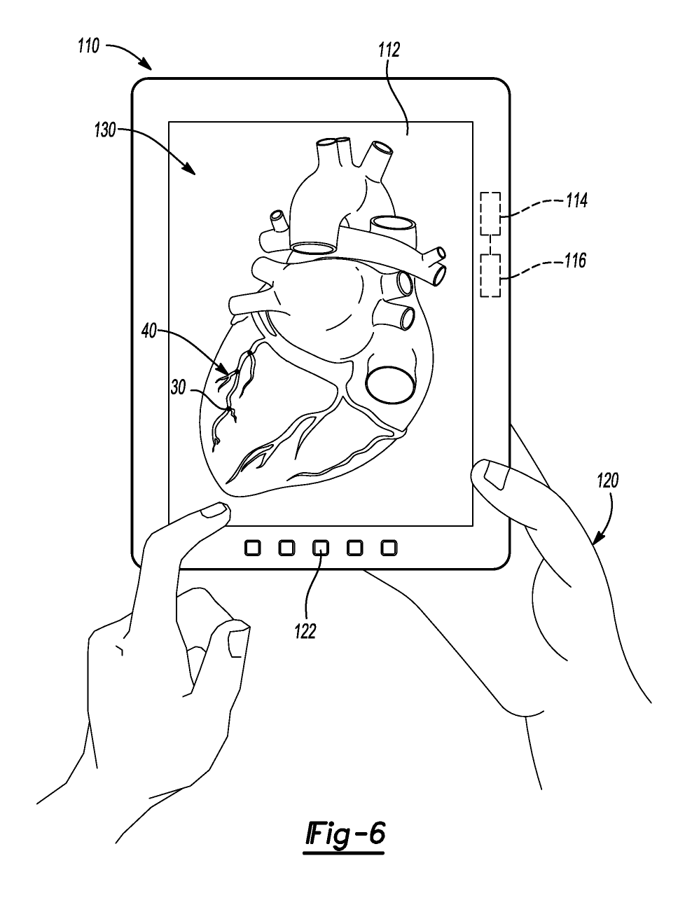

With continuing reference to FIGS. 1-5 and additional reference to FIG. 6, the plots illustrated in FIGS. 3, 4, and 5 can be displayed on a device 110. The device 110 can include a display device or screen 112. The display or screen 112 can be integrated into a hand-held device, as illustrated in FIG. 6, or can be a screen or display device of any appropriate system, such as a monitor for a laptop or desktop computer, which the device 110 may be. In addition, the device 110 can incorporate or be connected to a processing system or processor 114 and further be connected to or incorporate a memory system 116. The processor 114 can be any appropriate processor, such as a microprocessor or appropriate electronic based processing system. The processor 114 can be a software control general processor and/or an application specific processor (e.g., an application specific integrated circuit (ASIC)). The memory 116 can be an appropriate memory such as a solid state memory, a network access memory, a storage media memory, or the like. The memory system 116 can store instructions to be executed by the processor 114 and may also store information, such as information relating to diameters of the leads, including those illustrated in lines 90-96 on the plots discussed above. Further, the memory 116 can store or be able to access image data, such as image of the vasculature 40 or model images loaded into the memory.

As discussed above, the device 110 can be handheld, such as being portable or handheld by a user such that a hand 120 of a user can carry and operate the device 110. Various inputs, such as a touchscreen and/or access buttons 122 can also be used to access various portions of the device 110. For example, touching on the display 112 can identify the target location 30. It is understood that the access or inputs can also be used to change or augment the target, or other appropriate portion. Nevertheless, the display 112 can display image data and/or a model generated from image data and/or other information 130. The image data and/or model 130 can be used by a user to identify the target 30, select a lead for display, select a portion of the vasculature 40 to plot, and to display the plots as illustrated in FIGS. 3-5.

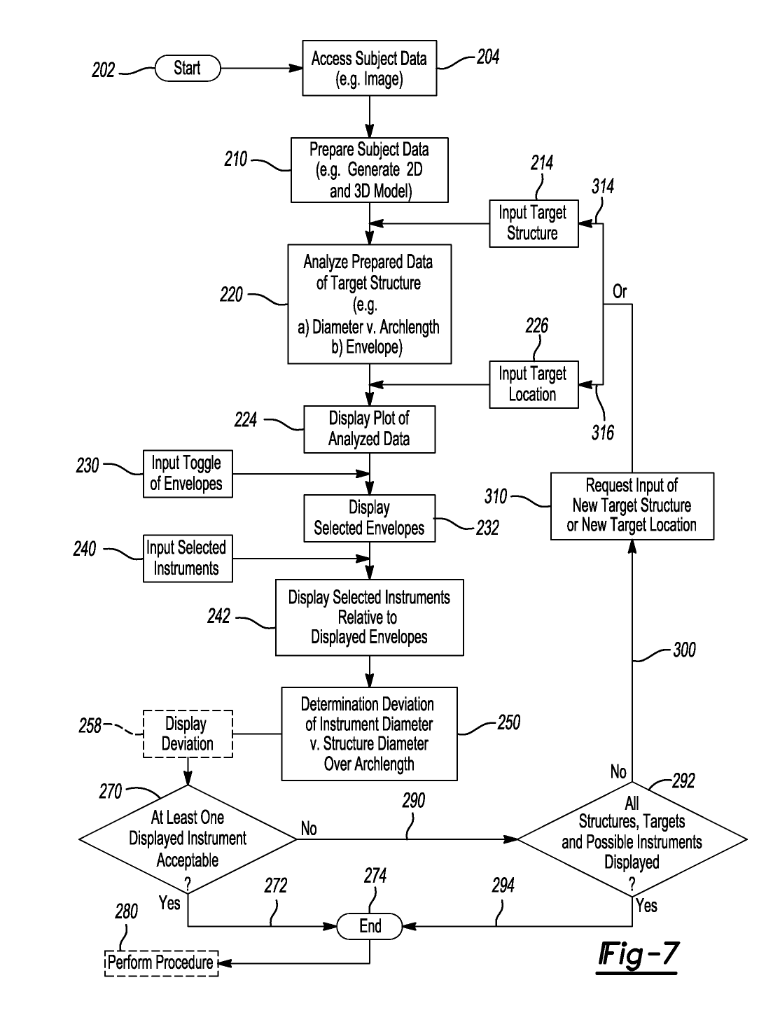

With continuing reference to FIGS. 1-6 and additional reference to FIG. 7, FIG. 7 illustrates a flow chart 200 that incorporates a method for selecting a target, generating information relating to a target, generating and/or displaying information relating to a lead and suggestions for a lead. For example, the steps in the flowchart 200 can be implemented in an algorithm that is executed by the processor 114. Further, the algorithm can be incorporated into instructions stored in the memory 116 that is executed by the processor 114. Accordingly, it is understood that the flowchart 200 illustrated in FIG. 7 can be incorporated into instructions that are executed by an electronic processor, such as a microprocessor or other processors, including those discussed above, for identifying or suggesting a lead as discussed further herein.

With continuing reference to FIG. 7, the flowchart 200 can include various algorithmic steps or instructions that can be executed by a processor system, as discussed above. Further, the flowchart 200 can include inputs that can be input directly from a user, such as a physician, and engineer, or the like, that may alter or identify specific steps being taken by a processor or to select branches in an algorithm. Accordingly, it is understood that the flowchart 200 illustrates a flow of instructions to be executed by a processor and/or inputs from a user.

The flowchart 200 can begin with start block 202. Initially, accessing subject data can occur in block 204. Subject data can include appropriate data, such as image data, drafting data, and other appropriate data. For example, subject data can include measurements or engineering drawing data for various structures, such as tubing. Further, subject data can include image data, such as venographic data, MRI data, or other appropriate image data of a subject. For example, and according to various embodiments including those discussed exemplarily herein in detail, the image data can include venographic image data. Venographic image data can include image data similar to that disclosed in U.S. patent application Ser. No. ______ (Attorney Docket No. 5074D-000086-US). Additionally, the venographic image data can include image data that is analyzed and used with the Cardio Guide® System, as noted above. Nevertheless, the image data can be acquired according to generally known techniques and/or any appropriate techniques to obtain image data of a subject.

After the image data is accessed, such as directly from an imaging system or stored in the memory 116, the subject data can be prepared in block 210. Preparing the subject data in block 210 can be any appropriate preparation. For example, a 2D model and/or a 3D model can be generated with the subject data. As discussed above, engineering drawings can also be used to generate a drawing model of a physical structure. Further, the subject data that may include image data can be analyzed to generate a 2D or 3D model of the imaged portion. According to various embodiments, the image data can be a venogram of a subject, such as a human patient, and including vasculature around and near the heart.

The image data of the subject can be analyzed and used to generate a 3D model of the vasculature of the subject. The 3D model can then be further analyzed to determine various geometric configurations, sizes, and the like of the imaged portion. For example, as is generally understood in the art, the venographic image data can be used to identify a diameter of a vessel at its centerline along its length. The diameter may be segmented along selected arc length portions of the vessel, and in appropriate increment, as discussed above. For example, an arc length may include about 0.1 millimeters (mm) to about 2 mm, further including about 0.5 mm to about 1 mm, and further including about 1 mm. The arc length can be used to identify or define a segment or portion of the vasculature to allow for a determination of the diameter at the arc length portion.

Accordingly, the preparation of the subject image data can be used to generate the 3D model, identify the selected arc length segments, and measuring or determining a diameter at each arc length. An input can then be received to input a target structure in block 214. The input can be a direct input, such as by a user using the input button 122 and/or touching the screen 112 of the device 110. For example, the image 130 can be an exemplary model generated with the image data, then the user can use a digit of the hand to identify a target structure. A target structure can include a length or section of the vessel 40 to be analyzed further. The target structure need not be a specific location within the structure, but can identify a whole structure, such as a branch from the coronary sinus or other appropriate structural portion.

After a target structure is input from block 214, an analysis of the prepared data of the target structure can be performed in block 220. The analysis of the prepared data in block 220 can include a determination of a diameter at various arc lengths of the structure. The diameter vs. the arc length can be determined for the entire length of the input structure or a selected portion of the input structure as selected by a user. The diameter can be determined based upon analysis of the image data, such as determining a pixel width of the structure in the image and correlating the number of pixels to a physical dimension, such as in millimeters, centimeters, or the like.

Further, the analysis of the prepared image data in block 220 can be compared to an envelope diameter at each arc length. As discussed above, and illustrated in FIG. 5, an envelope regarding the diameter of the structure can be calculated. In particular, as noted above, a structure may stretch under a certain load. Accordingly, the analysis of the prepared data of the target structure in block 220 can include analysis including various amounts of stretching, such as about 5%, 10%, or 20%. The diameter envelope relative to arc length can also be analyzed and/or generated in block 220. The analyzed and prepared data can also be stored in the memory system 116 for further analysis and display, as discussed further herein.

The analyzed prepared target data can then be displayed on the display device 112 in block 224. The display of the analyzed prepared data can be based upon or include an input target location from block 226. The input target location can include a direct input or recalled input of the target. Again, for example, the user can touch a portion of the display 112 to identify a specific target location, such as the target 30. The display plot of the analyzed data in block 224 can then be displayed on the display device 112, as illustrated in FIGS. 3-5. The target location 30 can be illustrated as a target location line 30′ on the selected plots. The target location illustrates the selected target location within the target structure input in block 214. Accordingly, a user can visually identify the target location diameter that relates to the arc length segment at the target location 30. Accordingly, the display 112 can display one or more of the selected plots of the diameter relative to length, as illustrated in FIGS. 3-5, including the diameter vs. arc length line 40a.

Also, as noted above, the envelope size relative to the diameter of the vessel can also be determined and illustrated, as exemplary illustrated in FIG. 5. Accordingly, input to the method can include inputting the toggle envelopes from block 230. The toggle input can include an automatic input, such as selectively displaying each of the determined envelope diameter or receiving input from a user, such as with the device 110. Accordingly, the display 112 can display selected envelopes in block 232. Further, as illustrated in FIG. 5, the display can display all of the selected envelopes or all or a multiple of envelopes simultaneously with a nominal measurement (e.g. the determined measurement of the arc length segment) in the display block 232.

Relative to the displayed diameter vs. arc length plots are displayed the instrument lines 90-94 or any selected number of instruments can be made. Accordingly, input to the system can include input of selected instruments in block 240. Again, it is understood that the input of selected instruments in block 240 can be input by a user directly or in real time, such as with the device 110, or substantially automatically. In various embodiments, the processor 114 in executing the instructions can recall selected or preferred or possible instruments for display relative to the plot. Additionally, a user may specifically select one or more instruments for displaying on the plots in the input block 240. Thus, display of the selected instruments relative to the displayed envelopes can be performed in block 242 on the display 112.

Again, as illustrated in FIGS. 3-5, the instrument lines 90-96 can be illustrated on the plots relative to the length axis 50 and the diameter axis 80 in the plotted arc length diameter of the vessel. Again, it is understood, that the instruments can have diameters and the display of the instrument lines 90-96 illustrate the diameter of the instrument. Accordingly, the instrument, as illustrated in FIGS. 3-5 may have a substantially constant diameter along its length to be positioned within the structure. It is understood, however, that the instrument may have a variable diameter along its length. For example, a distal tip of the instrument may have a diameter that is larger to assist in positioning and/or implantation of the instrument. Accordingly, it is understood that the plots can include a variation in the illustrated diameter of the instrument as well. For example, with reference to FIG. 5, the line 92 may include an enlarged portion 92a that illustrates that a distal tip of the instrument represented by line 92 is larger than a proximal portion thereof.

Once the instruments have been selected and/or displayed in blocks 240 and 242, a determination of a deviation of the instrument diameter vs. the structure diameter over the arc lengths can be determined in block 250. The determination of the deviation can include a least squares of the difference of the instrument diameter relative to the arc length segment illustrated by the vessel line 40a and/or 40b. For example, with reference to FIG. 4, a determination of the instrument represented by the line 90 can include a first difference or deviation that is a negative difference 252 and a second deviation or difference that is a positive difference 250. A summation of the deviations can be made for comparison of the various instruments by making these measurements and calculations. It can be selected to display and/or analyze the deviations in block 258. It is understood, however, that display of the deviations, such as a summation of the deviations, in block 258 is not required.

With continued reference to FIG. 7, the procedure and the system that is able to determine and selectively display deviations, as discussed above, can further include analysis of the displayed results. For example, after determination of the deviation of instrument diameter vs. the structure diameter over the arc lengths, a decision block 270 can be used to determine whether at least one of the displayed instruments is acceptable. The determination can be made by a user by viewing and analyzing the plots of the instruments relative to the diameters of the structure over the arc lengths. Further, a threshold can be used to automatically determine whether one or more of the displayed instruments is appropriate. For example, based upon a select amount of stretching, such as a 10% envelope diameter, the system can determine whether the determined deviation determined in block 250 is appropriate for maintaining any of the illustrated leads within the selected structure at the selected target location 30. For example, if the deviation includes a positive value of at least 1 mm, the system can suggest the lead that meets this deviation. It is understood that a positive deviation of 1 mm may indicate that the lead or selected instrument can have a diameter that is 1 mm greater, over the total arc length of the structure to the target location 30, than the structure, such as a vessel itself. This can ensure that the vessel will hold the lead in place relative to the target location 30.

If the determination that a displayed instrument is appropriate or acceptable, a YES path 272 can be followed to end the procedure or the analysis in block 274. It is understood that once an acceptable instrument is determined by following the YES path 272 that a procedure may be performed in block 280. The procedure performed may be separate from or following a determination of an appropriate instrument, but can include implantation of a cardiac lead, placement of a pulmonary lead, positioning of a scope within a tubing of a structure (e.g., a compressor or heat exchanger), or other appropriate procedure.

If the decision block 270 follows a NO path 290 that none of the displayed leads are acceptable, a second decision block 292 can be used to determine whether all possible target structures and target locations and instruments have been displayed. If it is determined that all target structures, all target locations, and that all possible instruments have been displayed, then a YES path 294 may be followed to the end block 274. In this instance, however, performing a procedure 280 may not occur as no target structure, target location, or possible instrument has been determined to be appropriate. It is understood, however, that further image data may be acquired, a larger library of possible instruments, or other analysis may occur to perform a procedure, as selected.

Nevertheless, if it is determined that all target structures, all target locations, or all possible instruments have not been displayed, a NO path 300 can be followed to a request for input of a new target structure and/or a new target location in block 310. It is understood that the input of a new target structure or a new target location can be made by a user, such as a surgeon, inputting instruction into the system 110 or an automatic selection of a new target location or a new target structure. For example, a user or system can identify a new branch vessel to select for analysis by the procedure 200 for possible placement of an instrument. Further, the system or user can select a new location, such as a more distal location, for implantation of a lead. Accordingly, a request for input can lead to an alternative of input target structure in block 214 or input target location in block 226 by following input path 314 and/or 316, respectively. It is understood that the flowchart 200 can be executed in such a manner until stopped by the system, stopped by the user, or stopped by the algorithm based upon having determined that all possible structures, target locations, and instruments have been displayed or analyzed and all have been determined to be not acceptable.

Accordingly, as discussed above, a user can use the method 200, such as executed by the processor 114, to assist in illustrating and/or suggesting possible or acceptable lead instruments for selected target structures and/or selected target locations as input in blocks 214 and/or 226, respectively. The results can be displayed on plots, as exemplary illustrated in FIGS. 3-5, for viewing and comprehension by a user or the system. Accordingly, a user can efficiently identify, such as by viewing the plots, an instrument that may be appropriate for a measured diameter of a structure over a selected arc length of a structure. The method may also be used to instruct a processor to suggest leads based on appropriate structure to instrument relationships.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

What is claimed is:

1. A system to illustrate an instrument geometry relative to a selected geometry of a structure, comprising:
a display device configured to display at least one of a geometric configuration of an instrument or a geometric configuration of a structure; and
a processor device configured to execute instructions to:
access structure data;
analyze the accessed structure data to determine the geometric configuration of at least a portion of the structure; and
generate a plot of the geometric configuration of at least the portion of the structure relative to a geometric configuration of the instrument.

2. The system of claim 1, wherein the display device is configured to display both the geometric configuration of the instrument and the geometric configuration of the structure.

3. The system of claim 2, wherein the display device is configured to simultaneously display both the geometric configuration of the instrument and the geometric configuration of the structure over a selected length of both the instrument and the structure.

4. The system of claim 3, wherein the geometric configuration includes a diameter of the structure.

5. The system of claim 4, wherein the diameter of the structure includes a discrete diameter at discrete arclengths of the structure.

6. The system of claim 1, wherein analyze the accessed structure data to determine the geometric configuration of at least the portion of the structure includes determining a plurality of arclength segments along a centerline of the structure and determining a diameter within each arclength segment of the plurality of arclength segments.

7. The system of claim 1, wherein the accessed structure data includes a venogram of a vasculature.

8. The system of claim 1, wherein the geometric configuration of the instrument includes a stored diameter of the instrument to be compared to a determined geometric configuration of the structure.

9. A system for illustrating relationships, comprising:
a memory system configured to store a geometric configuration of at least one instrument;
an input system configured to allow input from a user;
a processor system configured to execute instructions to:
prepare image data of a structure;
analyze the prepared image data to determine geometric configuration of the structure; and
plot the determined geometric configuration of the structure relative to the stored geometric configuration of the at least one instrument; and
a display device to display the plot.

10. The system of claim 9, wherein the at least one instrument includes a plurality of instruments.

11. The system of claim 10, wherein the plot of the determined geometric configuration of the structure includes a plot of a plurality of diameters of the structure, wherein each diameter is at a specific arclength of the structure.

12. The system of claim 11, wherein the processor system is further configured to determine an overall deviation of the geometric configuration of the structure compared to the geometric configuration of at least one of the plurality of instruments.

13. The system of claim 9, wherein prepare the image data includes generating a three-dimensional model of the structure based on the image data.

14. The system of claim 9, further comprising:
the processor system further configured to determine a stretch percent of the structure and plot the stretch percent.

15. A method of illustrating relationships, comprising:
receiving an input of at least one geometric feature of at least one instrument;
receiving data regarding a structure;
preparing the data for analysis;
analyzing the prepared data to determine at least one geometric configuration of the structure;
illustrating the at least one geometric feature of the at least one instrument as a first plot;
illustrating the determined at least one geometric configuration of the structure as a second plot.

16. The method of claim 15, further comprising:
comparing the first plot and the second plot.

17. The method of claim 16, wherein comparing the first plot and the second plot includes determining a deviation of an instrument diameter relative to a structure diameter over cumulative arc lengths of the structure.

18. The method of claim 15, further comprising:
illustrating the first plot and the second plot on the same set of axes.

19. The method of claim 15, wherein receiving the input of at least one geometric feature of at least one instrument, includes receiving the of geometric feature for a plurality of instruments;
wherein the first plot includes a plot line relating to each instrument of the plurality of instruments.

20. The method of claim 15, further comprising:
determining a second geometric configuration of the structure based on a stretch factor of the structure; and
illustrating the second geometric configuration of the structure as a third plot.

21. The method of claim 15, further comprising:
receiving an input to define the structure.

22. The method of claim 21, further comprising:
receiving an input to define a target location within the structure.

23. The method of claim 15, wherein the data regarding the structure includes image data of the structure.

24. The method of claim 23, wherein preparing the data for analysis includes generating a three-dimensional model of the structure.
